(19) United States
(12) Patent Application Publication
(10) Pub. No.: US 2024/0316215 A1
(43) Pub. Date: Sep. 26, 2024

(54) CONTROLLED DRUG RELEASE MATERIAL

(71) Applicant: ADURO MATERIAL AB, Uppsala (SE)

(72) Inventor: Håkan ENGQVIST, Uppsala (SE)

(21) Appl. No.: 18/291,635

(22) PCT Filed: Jul. 18, 2022
(86) PCT No.: PCT/SE2022/050722

§ 371 Date: Jan. 24, 2024

(30) Foreign Application Priority Data
Aug. 8, 2021 (SE) 2130219-5

(51) Int. Cl. A61K 47/69; A61K 47/52; A61P 35/00

(52) U.S. Cl. CPC A61K 47/6949; A61K 47/52; A61P 35/00

(57) ABSTRACT

The present invention relates to pharmaceutical compositions comprising an inorganic salt and hydroxyurea or a pharmaceutically acceptable salt thereof. The composition is in the form of doped crystals so that the hydroxyurea, or the pharmaceutically acceptable salt thereof, is included in the crystal lattice of the inorganic salt. The invention also relates to use of a pharmaceutical composition according to the invention as a medicament.

### FIELD OF THE INVENTION

This invention relates to pharmaceutical compositions comprising an inorganic salt and hydroxyurea or a pharmaceutically acceptable salt thereof. The invention also relates to use of a pharmaceutical composition according to the invention as a medicament.

### BACKGROUND

Drug delivery describes methods and approaches to deliver drugs and other active ingredients to their site of action within an organism. A lot of research in recent decades has focused on ‘controlled and/or targeted drug delivery’, i.e. the ability to control the delivery and release of drugs and pharmaceuticals to the desired place. ‘Drug release’ relates to a process wherein drug solutes migrate from a delivery vehicle to a release medium. Controlled drug release and targeted drug delivery can reduce systemic toxicity of drugs and pharmaceuticals, such as chemotherapeutics, by restricting drugs and pharmaceuticals to the target site, such as organ, and increasing the local concentration.

Bone void fillers and bone cements have been used for various applications related to bones for several years. It is a reliable material and used in routine procedures today. Bone void fillers and bone cements could potentially be used in the treatment of different bone diseases, such as fractures, osteoporosis, osteoarthritis and different bone cancers. Ewing sarcoma is a type of bone cancer wherein tumours are formed inside the bone or in the soft tissue around the bone. It occurs most common in children and young adults.

Chemotherapeutics are chemical substances used to treat or cure cancer. They are generally highly potent substances that has to be handled with care. Direct contact with chemotherapy drugs should be avoided due to the health risks associated with that.

U.S. Pat. No. 10,722,596 B2 discloses composites comprising a metal carbonate and organic agent included within a crystal lattice of the metal carbonate.

US 2003/082240 A1 discloses an insulin formulation with porous, spherical calcium carbonate composed if trabeculate or needle-shaped crystals, or an aggregation of the parallel intergrowth of these forms, as its carrier.

In the prior art there is a need for a pharmaceutical composition that both enables a controlled release of the drug and also a safe handling of the drug prior to use.

### SUMMARY OF THE INVENTION

The present invention relates to a pharmaceutical composition, a pharmaceutical composition for use as a medicament, and a method of manufacturing a pharmaceutical composition.

In a first aspect of the invention there is a pharmaceutical composition comprising an inorganic salt and hydroxyurea, or a pharmaceutically acceptable salt thereof.

The inorganic salt selected from the group consisting of calcium carbonate, magnesium carbonate, calcium phosphate, and any combination thereof. The composition is in the form of doped crystals so that the hydroxyurea, or the pharmaceutically acceptable salt thereof, is included in the crystal lattice of the inorganic salt.

In one embodiment of the invention the pharmaceutical composition is characterized by an X-ray powder diffractogram recorded using Cu—K_{α} radiation, which is devoid of peaks at positions corresponding to hydroxyurea, or the pharmaceutically acceptable salt thereof.

In one embodiment of the invention the inorganic salt is selected form the group consisting of: calcite, aragonite, vaterite, calcium carbonate monohydrate, magnesium calcite, magnesite, magnesium carbonate monohydrate, nesquehonite, dolomite, hydroxyapatite, β-tricalcium phosphate, α-tricalcium phosphate, calcium pyrophosphate, tera-calcium phosphate, monetite, octo-calcium phosphate, and brushite.

In one embodiment of the invention the pharmaceutical composition comprises hydroxyurea and calcite.

In one embodiment of the invention the concentration of hydroxyurea is 2-20 μg hydroxyurea per mg calcite.

In one aspect of the invention there is a pharmaceutical composition for use as a medicament.

In one embodiment of the invention there is a pharmaceutical composition for use in the treatment of cancer.

In one embodiment of the invention there is a pharmaceutical composition for use in the treatment of Ewing sarcoma.

In one aspect of the invention there is a method for producing a pharmaceutical composition, wherein the method comprises the steps of:
dissolving hydroxyurea, or a pharmaceutically acceptable salt thereof, in a solvent and adding an inorganic salt forming a solution, wherein the inorganic salt is selected from the group consisting of calcium carbonate, magnesium carbonate, calcium phosphate, and any combination thereof;
carbonizing the formed solution forming a carbonized solution; and
precipitating doped crystals from the carbonized solution from the carbonized solution, wherein the hydroxyurea, or the pharmaceutically acceptable salt thereof, is included in the crystal lattice of the inorganic salt.

### Abbreviations

API—active pharmaceutical ingredient;
DMEM—Dulbecco's modified eagle medium;
EC₅₀—also referred to as ‘potency’, the concentration of API or drug that is required to produce 50% of maximum response;
HPLC—high-performance liquid chromatography;
PBS—phosphate buffered saline;
SEM—scanning electron microscopy;
UV—ultraviolet
XRD—X-ray diffraction

### LIST OF FIGURES

FIG. 1A-C shows diffractograms for embodiments according to the invention: A) shows a diffractogram for calcium carbonate synthesized without dopant, B) shows a diffractogram for calcium carbonate synthesized with 400 mM dopant, and C) shows a diffractogram for calcium carbonate synthesized with 800 mM dopant. The peaks marked with ▪ belongs to vaterite and the peaks marked with ● belongs to calcite;

FIG. 2A-F shows SEM micrographs of embodiments according to the invention: A) calcium carbonate crystals without dopant; B) calcium carbonate crystals without dopant after 2 weeks in PBS solution; C) calcium carbonate crystals synthesized with 400 mM dopant; D) calcium carbonate crystals synthesized with 400 mM dopant after 2 weeks in PBS solution; E) calcium carbonate crystals synthesized with 800 mM dopant; and F) calcium carbonate crystals synthesized with 800 mM dopant after 2 weeks in PBS solution;

FIG. 3A-D shows SEM micrographs of embodiments according to the invention: A) hydroxyapatite crystals synthesized at pH 12 and 40° C.; B) hydroxyapatite crystals synthesized at pH 8.5 and 40° C.; C) hydroxyapatite crystals synthesized at pH 12 and 70° C.; and D) hydroxyapatite crystals synthesized at pH 8.5 and 70° C. All crystals were synthesized with a dopant concentration of 0.07 mg/ml. The scale bars represent 1 μm;

FIG. 4A-F shows images according to embodiments of the invention: A)-F) are images of crystals in acidified DMEM cell media at T=0 h, 1 mg/ml incubated at 37° C. at different pH-values: A) pH=1; B) pH=2; C) pH=3; D) pH=4; E) pH=5; and F) pH=6.5;

FIG. 5 shows dissolution of calcium carbonate crystals according to embodiments of the invention measured by absorbance at different time points and at different pH-values: (x) pH=5 and T=1 h; (∘) pH=5 and T=24 h; (▴) pH=6 and T=1 h; (●) pH=6 and T=24 h; (♦) pH=6.5 and T=1 h; and (▪) pH=6.5 and T=24 h;

FIG. 6 shows drug released from pharmaceutical compositions according to the invention;

FIG. 7 is graph from a cell viability test using three different cell concentrations: (▪) 1×10⁴; (●) 2×10⁴; and (▴) 4×10⁴ cells/well;

FIG. 8A-B shows graphs from cell viability tests after being treated with pharmaceutical compositions according to embodiments of the invention: A) shows the results from 20,000 cells/well, and B) shows the results from 40,000 cells/well. The cells were treated with the pharmaceutical composition at concentrations of 50 mg/mL; 10 mg/mL; 5 mg/mL; and 1 mg/mL, for each concentration doped (+) crystals and non-doped (−) crystals were tested; and

FIG. 9A-C shows graphs from cell viability tests after being treated with crystals according to embodiments of the invention: A) shows the results from 10,000 cells/well, B) shows the results from 20,000 cells/well, and C) shows the results from 40,000 cells/well. The cells were treated with 5 mg/mL, and 2.5 mg/mL, for each concentration doped (+) crystals and non-doped (−) crystals were tested. D1 denotes measurement at day 1 and D3 denotes measurements at day 3. The staples show mean±SD, n=3, *p<0.05, **p<0.01 in comparison with respectively control group.

FIG. 10 is a flow chart illustrating a method for producing a pharmaceutical composition according to an embodiment.

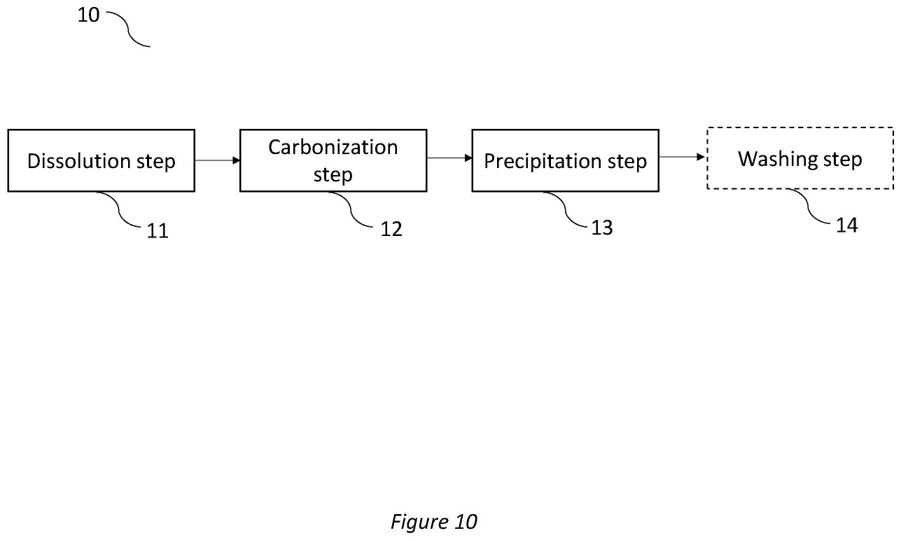

### DETAILED DESCRIPTION

Targeted drug delivery, or targeted drug release materials, is interesting for many reasons not the least for its potential ability to reduce side effects since a lesser amount of drug can be administrated when it is administrated directly to or close to the desired site inside the human or animal and still achieve the same therapeutic effect as compared to systemic administration of the drug at a comparatively higher systemic amount. The present invention relates to a pharmaceutical composition for targeted drug delivery. The composition comprises an active pharmaceutical ingredient (API) and an inorganic salt wherein the API act as a dopant and as such is enclosed within the crystal lattice of the inorganic salt. Local delivery of APIs can allow for effective treatment of different conditions, such as for example cancer. One example is effective treatment of cancer in connection with bone void filling after removal of bone cancer.

Different inorganic salts such as calcium carbonate, magnesium carbonate, and calcium phosphates have been widely studied for their use in different drug delivery applications. The most common way of synthesizing calcium carbonate, magnesium carbonate and calcium phosphates, is different variations of precipitation methods, which are usually free from organic solvents, fairly simple and have an overall low cost. Controlled synthesis of calcium carbonate is not only cost effective but it also makes it possible to modify the size, morphology, shape and functionalization. For drug delivery applications crystal morphology is one concern, together with drug loading efficiency, since the morphology of the delivery vehicle determines the dissolution rate, surface energy, and potential for direct cell uptake (passive drug targeting). Previous studies have shown that it is possible to synthesize pH responsive hybrid nano- and microparticles with variating sizes and shapes while adding drugs by the use of adsorption onto the particle surfaces. One example was reported to incorporate small molecules (amino acids) into a single calcite crystal. However, in that example an increasing amount of enclosed amino acid resulted in change of morphology, i.e., rounded corner. It is known that impurities can inhibit crystal growth by blocking active sites such as Ca²⁺ and CO₃²⁻. Amino acids are known to be highly adsorbent to calcite where the calcium ions on the calcite surface interact with the carboxyl group and the carbonate group interact with the amino group.

In a first aspect of the invention there is a pharmaceutical composition comprising an inorganic salt and hydroxyurea, or a pharmaceutically acceptable salt thereof. The inorganic salt selected from the group consisting of calcium carbonate, magnesium carbonate, calcium phosphate, and any combination thereof. The composition is in the form of doped crystals so that the hydroxyurea, or the pharmaceutically acceptable salt thereof, is included in the crystal lattice of the inorganic salt.

Herein the word ‘dopant’ is used to describe a molecule, substance, or active pharmaceutical ingredient (API) enclosed, or included, in the crystal lattice of an inorganic crystal. The word ‘doped’ is used to describe a crystal, e.g., a single crystal, comprising at least one dopant. In one embodiment, the inorganic crystal is a single crystal. When a molecule or substance is enclosed, or included, in the crystal lattice it does not give rise to any peaks in a powder X-ray diffractogram recorded of the inorganic crystal. In one embodiment of the invention, the pharmaceutical composition is characterized by an X-ray powder diffractogram recorded using Cu—K_{α} radiation which is devoid of peaks at positions corresponding to hydroxyurea, or a pharmaceutically acceptable salt thereof. Additionally, a doped crystal preferably has the same or essentially the same morphology as a non-doped crystal.

In one embodiment of the invention the pharmaceutical composition comprises urea or a pharmaceutically acceptable derivate thereof. In such case the urea or pharmaceutically acceptable derivate thereof is a dopant and hence enclosed in the crystal lattice of the inorganic salt.

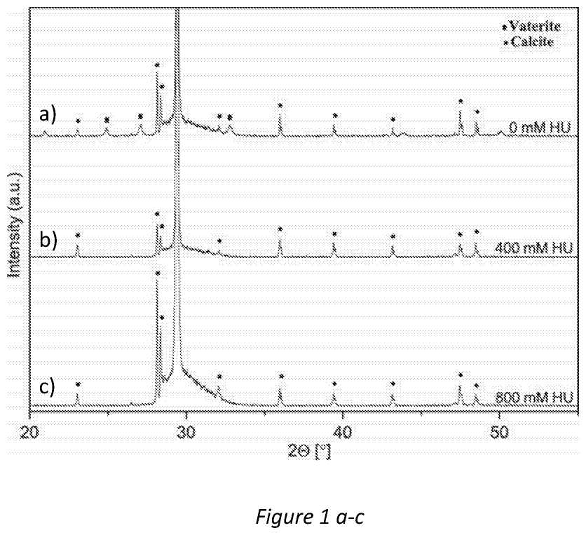

Examples of powder X-ray diffractograms of pharmaceutical compositions according to the invention can be seen in FIGS. 1B and C. As can be seen no peaks belonging to the API, hydroxyurea in this example, can be seen in the diffractograms. What however can be seen in diffractograms of compositions according to the invention is a broadening of the peaks. This can for example be seen in FIGS. 1B and C in comparison with FIG. 1A that shows a diffractogram from a non-doped sample. The data showed in FIGS. 1B and C can be used to estimate the degree of crystallinity, for FIG. 1A slightly lower crystallinity was estimated to the doped crystals compared to the undoped crystals, 83% compared with 87%. Hence, a composition according to the invention may show a lower degree of crystallinity as compared to non-doped crystals of the same type.

The API enclosed in the crystal, or the crystal doped with the API or substance enables a controlled and/or targeted delivery of the molecule, i.e., the API, to a site in a human or animal.

In one embodiment the active pharmaceutical ingredient in the pharmaceutical composition is urea, or a pharmaceutically acceptable derivate thereof. In one embodiment the derivate of urea is hydroxyurea. A pharmaceutical composition according to the invention can additionally, or alternatively, comprise a pharmaceutical salt of urea or its derivates. In one embodiment of the invention the active pharmaceutical ingredient is hydroxyurea. Additionally, a pharmaceutical composition according to the invention can additionally, or alternatively, comprise a pharmaceutical salt of hydroxyurea.

Urea, or ammonium carbamate, having the chemical formula (NH₄)(H₂NCO₂) is a salt comprising the ammonium cation (NH⁴⁺) and the carbamate anion (NH₂COO⁻). Hydroxyurea, or hydroxycarbamide, has the chemical formula CH₄N₂O₂.

Calcium carbonate (CaCO₃) is an inorganic material that can be found in nature, where it is a key part of life as many organisms use CaCO₃ for storing ions and molecules. Calcium carbonate is interesting for different biomedical applications due to its biocompatibility, large specific area, and pH-responsiveness. These properties have resulted in an extensive interest of CaCO₃ in the research fields of pharmaceuticals and biomaterials where CaCO₃ could act as a target delivery system. CaCO₃ have three different polymorphs, i.e., calcite, aragonite, and vaterite. However, the most studied polymorph of CaCO₃ is calcite, which has a hexagonal crystal structure and is the most thermodynamically stable polymorph. Calcium carbonates can be used as bone void filler, i.e., filling of critical sized bone defects.

Magnesium carbonate or calcium/magnesium carbonates are also suggested for drug delivery and bone void filler applications. So far loading of active molecules into the crystal structure, without altering the morphology has not been described before.

Calcium phosphates are extensively used in both pharmaceutical and medical device applications. There are several calcium phosphates that can be used for various medical applications e.g., hydroxyapatite, beta-tricalcium phosphate, alpha-tricalcium phosphate, calcium pyrophosphate, tetra-calcium phosphate, brushite, etc., and the material can be delivered as a cement paste, granules and grains. The chemistry of the calcium phosphates allows the material to be used as cement, although mechanically weaker than Portland cement. Calcium phosphates can be found in e.g., bone void filler applications where the material is slowly resorbed in vivo. As for calcium carbonate, the calcium phosphates are proposed to be used in drug delivery applications. Controlled and/or targeted release is proposed to be achieved via surface adsorption or loading of the API into the pore system of the material.

In one embodiment of the invention the inorganic salt is a calcium carbonate, a magnesium carbonate, or a calcium phosphate or a mixture of those. Such inorganic salts can dissolve in vivo whereby the API is released in a controlled manner.

Inorganic crystals, such as calcium carbonate, magnesium carbonate, and calcium phosphate, are stable at neutral pH but they are sensitive to acidic pH. They can therefore be used in a pH-controlled drug delivery system. Cancer tumors generally lowers the pH of the tissue surrounding the cancer. Therefore, if a pharmaceutical composition according to the invention is administrated to a tumor site or vicinity of a tumor by for example injection into the tissue, the acidic environment at the site may lead to dissolution of the inorganic crystals and release of the API at the site.

The calcium carbonate in a pharmaceutical composition according to the invention may be any of: calcite (CaCO₃), aragonite (CaCO₃), vaterite (CaCO₃), calcium carbonate monohydrate (CaCO₃×H₂O), or magnesium calcite (Mg—CaCO₃). The magnesium carbonate in a pharmaceutical composition according to the invention may be any of: magnesite (MgCO₃), magnesium carbonate monohydrate (MgCO₃×H₂O), nesquehonite (MgCO₃×3H₂O), or dolomite (CaMg(CO₃)₂). The calcium phosphate in a pharmaceutical composition according to the invention may be any of: hydroxyapatite (Ca₅(PO₄)₃(OH)), β-tricalcium phosphate (β-Ca₅(PO₄)₂), α-tricalcium phosphate (α-Ca₅(PO₄)₂), tera-calcium phosphate (Ca₄(PO₄)₂O), monetite (Ca(PO₃OH), octo-calcium phosphate (Ca₈H₂(PO₄)₆×5H₂O), or (CaHPO₄×2H₂O).

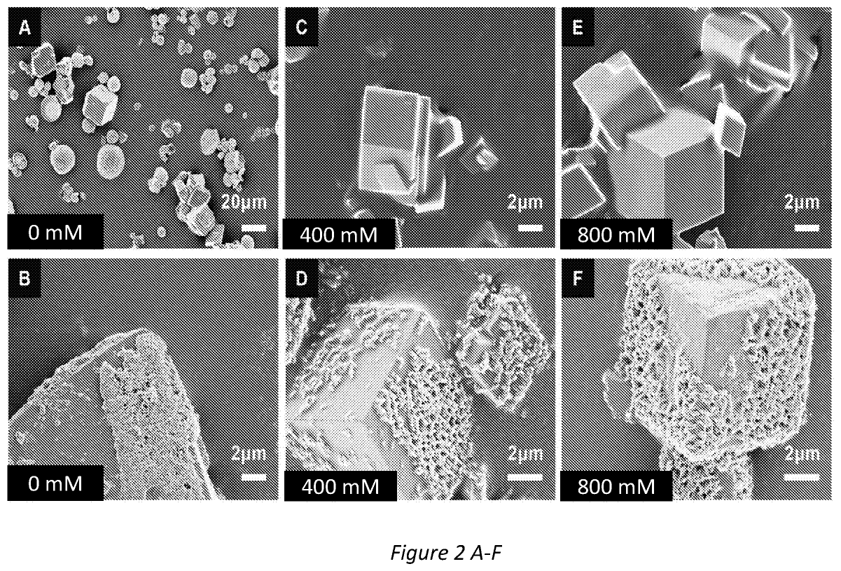

Inorganic crystals in a pharmaceutical composition according to the invention maintain a perfect or almost perfect crystal structure, as for example can be seen in FIGS. 2C and E that shows SEM images of compositions according to the invention comprising calcium carbonate doped with hydroxyurea. As can be seen in the images the morphology of the crystals is the same as for non-doped calcite crystals, FIG. 2A.

In one embodiment of the invention, the pharmaceutical composition comprises hydroxyurea and calcite.

Hydroxyurea, also known as hydroxycarbamide, is a cytotoxic, anti-proliferative drug used to treat rapidly dividing diseases, such as sickle cell, leukemia, bone cancer, and breast cancers. Hydroxyurea comprises amino groups and lies between the amino acids glycine and aspartic acid in size, see below:

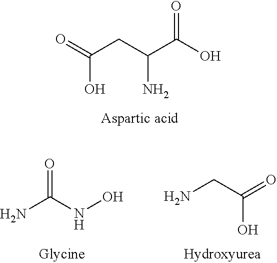 [SMILES: NC(CC(=O)O)C(=O)O ; NC(=O)NO ; NCC(=O)O]

In one embodiment of the invention, the concentration of API, e.g., hydroxyurea, in the pharmaceutical composition is 2-20 μg API per mg inorganic crystal, e.g., calcite. The concentration of API, e.g. hydroxyurea, in the pharmaceutical composition may also be lower such as 0.5-10 μg API per mg inorganic crystal, or 0.5-5 μg API per mg inorganic crystal, or 2 μg API per mg inorganic crystal. In one embodiment the pharmaceutical composition comprises 2 μg hydroxyurea per mg calcium carbonate. In one embodiment, a majority or essentially all of the API is enclosed, or included, in the inorganic crystal. A pharmaceutical composition according to the present embodiment may achieve growth arrest of a tumor since it comprises a clinical feasible dosage of the API of at least 5 mg/kg body weight and per day.

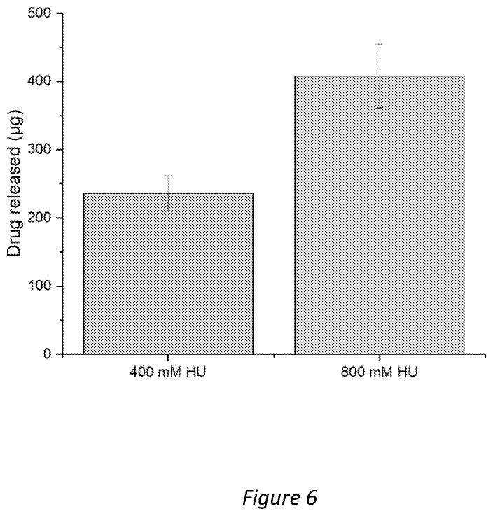

The amount of API in a pharmaceutical composition can, for example, be measured by a drug release test. In such a drug release test the inorganic crystals are dissolved by an acid and the amount of API is measured using for example HPLC. FIG. 6 shows the results from such a test, as can be seen in the figure all API that was incorporated in the inorganic crystals have been released.

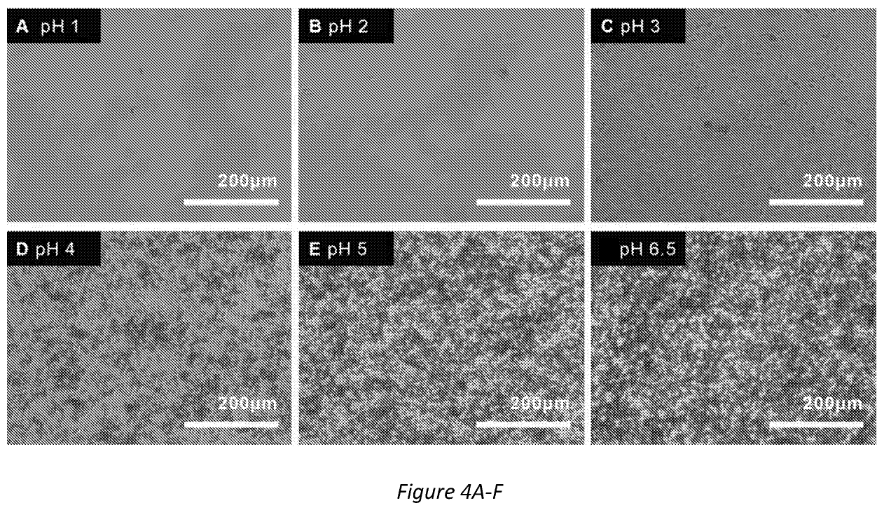

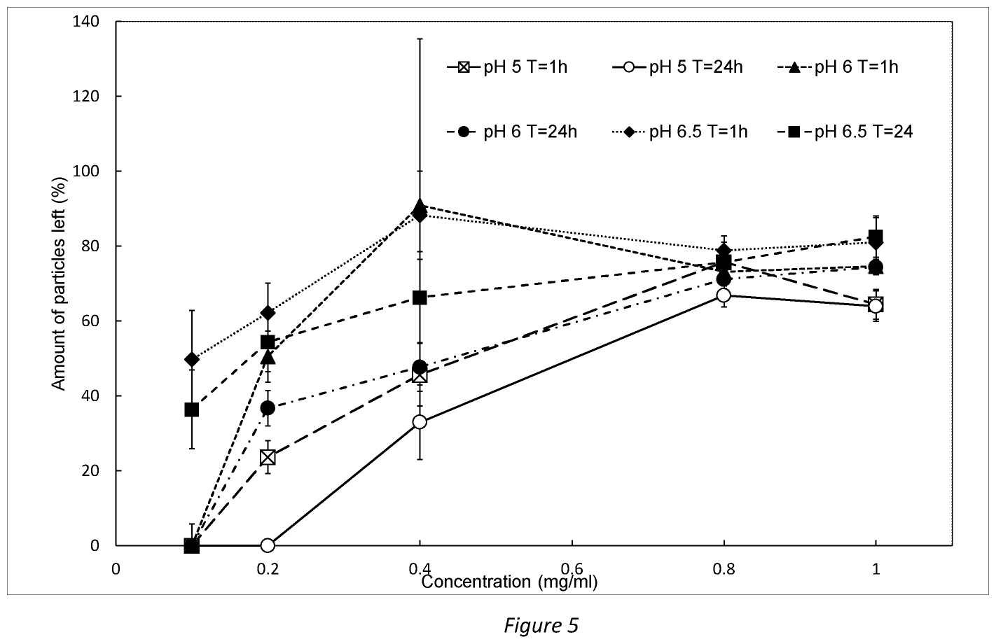

In order for a pharmaceutical composition according to the invention to be useful as a medicament, the crystals have to dissolve in vivo. The physiological pH value is approximately 7.3-7.5. At this pH value there were almost no dissolution of the doped crystals (data not shown). However, at a tumor site the pH is lower. The dissolution of pharmaceutical compositions according to the invention was measured at pH-values between 5 and 6.5 after 1 h and after 24 h, the results are shown in FIG. 5. As can be seen in FIG. 5 almost full dissolution of the doped crystals were reached after 1 hour in pH 5 and 6. FIG. 4A-F additionally shows images of crystals incubated in cell media at different pH values. As can be seen in FIG. 4A-C is that under pH 4 full dissolution of the compositions are achieved.

Additionally, essentially no drug substance is released over time at physiological pH (data not shown). Hence, a pharmaceutical composition according to the invention demonstrated an excellent pH responsiveness where no or essentially no API will leak at physiological pH for a pro-longed time period. As such, pharmaceutical compositions according to the invention may be suitable for direct injection in the vicinity of tumors where they will dissolve due to the low pH and release the API.

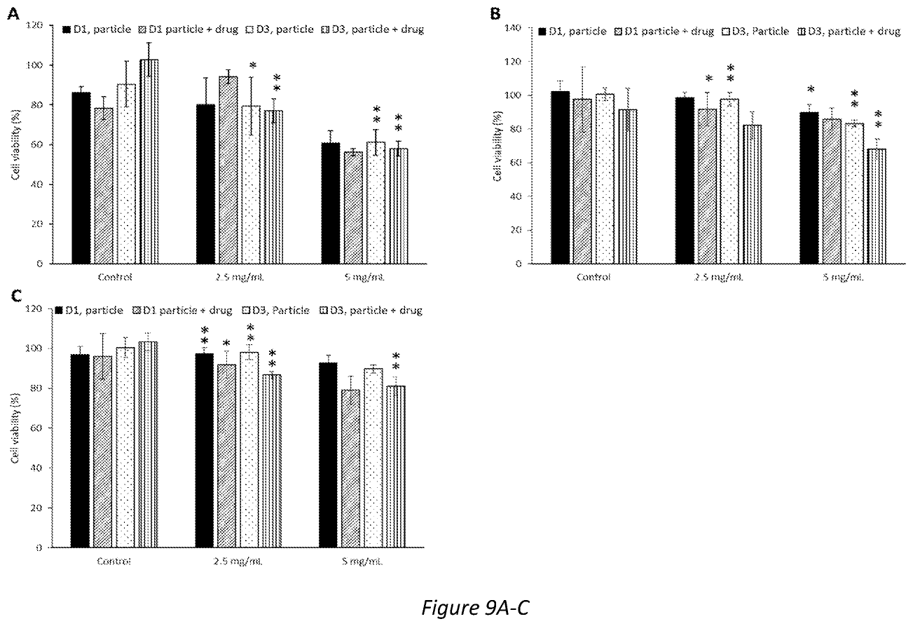

It is an advantage that a pharmaceutical composition according to the invention is essentially nontoxic at physiological conditions. Results from cell viability measurements for a pharmaceutical composition according to the invention and a control group can be seen in FIG. 9A-C. As can be seen the observed cell death was similar in both groups: doped and non-doped.

Further, FIGS. 2B, 2D, and 2F shows doped (FIGS. 2D and 2F) and non-doped (FIG. 2B) calcium carbonate crystals that have been stored for 2 weeks in PBS solution. As can be seen in the SEM images, hydroxyapatite was formed on the surface after two weeks of storage in PBS for all samples.

A pharmaceutical composition according to the invention can be formulated in several different ways depending on the desired route for administration. For example: the pharmaceutical composition can be directly injected using a syringe, or the pharmaceutical composition may be mixed with, for example, carboxymethylcellulose forming a gel. The relative ratio in weight for a gel may be up to 50:50 doped crystals to gel, preferably a ratio of 40:60 or less. The term ‘pharmaceutical composition’ refers here to a bone void filler comprising an API such as hydroxyurea for example. A pharmaceutical composition according to the invention may thus for example be a calcium phosphate bone void filler comprising hydroxyurea-doped calcium carbonate crystals.

A pharmaceutical composition according to the invention may be mixed with a resorbable bone cement or bone void filler precursor powder, e.g., calcium sulphate or calcium phosphate, as a filler in the range of 30 wt % of the precursor powder. In such case the setting pH of the cement should preferably be above ˜6. Calcium sulphate cements, α-tri calcium phosphate-based cements and tetracalcium phosphate-based cements are suitable. For a combined fast and slow release, the API can be added directly to a formulation comprising a bone cement or bone void filler and an inorganic salt. In such case the amount of drugs should be balanced to achieve a high local concentration, e.g., 20 wt %.

A pharmaceutical composition according to the invention can be combined with resorbable granules. The relative ratio between the pharmaceutical composition and resorbable granules can vary between 10:90 to 90:10. The resorbable granules may be solid or porous. They may be composed of any combination of carbonate, phosphate, or sulphates. Such a formulation of a pharmaceutical composition according to the invention and resorbable granules may be delivered as a putty with, for example, carboxymethylcellulose as carrier. For a combined fast and slow release, the API can be added directly to a formulation. In one embodiment, there is a formulation comprising hydroxyurea-doped calcium carbonate crystals and ß-tri calcium phosphate. In further embodiments such composition may additionally comprise free hydroxyurea. The ratio between the pharmaceutical components may be 20-16 hydroxyurea:75-71 β-tri calcium phosphate granules:5-1 hydroxyurea doped calcium carbonate. In still further embodiments, resorbable granules with API doped inorganic crystals inside the granules can also be found. The amount of API doped crystals in the granules may be up to 80 wt %, preferably less than 50 wt %, more preferably less than 30 wt %, or even more preferably less than 10 wt %.

In one embodiment, the pharmaceutical composition comprises urea as API. In another embodiment, the pharmaceutical composition comprises a derivative of urea as API or multiple, i.e., at least two, different derivatives of urea as API. In a further embodiment, the pharmaceutical composition comprises a salt of urea as API or multiple different salts of urea as API. In yet other embodiments, the pharmaceutical composition comprises urea and at least one derivative of urea as API, urea and at least one salt of urea as API, at least one derivative of urea and at least one salt of urea as API, or urea, at least one derivative of urea and at least one salt of urea as API. In one embodiment, the pharmaceutical composition comprises hydroxyurea as API and at least one salt of hydroxyurea.

The inorganic crystals in a pharmaceutical composition according to the invention is generally stable at neutral pH, and sensitive to acidic pH. This enables pH-triggered dissolution of the crystals and hence pH-triggered release of the API. An advantage with this is that this increases the safety of the composition. A pharmaceutical composition according to the invention may thus be used to store APIs in a safe way. Since the APIs are included, or enclosed, in the crystal lattice the pharmaceutical composition can be handled without the risk or with a minor risk of side effects caused by the API.

An aspect of the invention relates to a pharmaceutical composition according to the invention for use as a medicament.

A pharmaceutical composition according to the invention may, for example, be used in bone void filler applications for local controlled delivery of drugs in e.g., bone voids after removing bone tumors. In one embodiment there is a pharmaceutical composition according to the invention for use in the treatment of cancer. The cancer may for example be Ewing sarcoma. The present invention also relates to the use of a pharmaceutical composition according to the invention for the manufacture of a medicament for treatment of cancer, such as Ewing sarcoma.

A pharmaceutical composition according to the invention may additionally be used for local delivery of hydroxyurea, or a pharmaceutically salt thereof, to tumors outside the bone.

A further aspect of the invention relates to a method for treatment of cancer. The method comprises administering a pharmaceutical composition according to the invention to a subject, preferably a subject suffering from cancer or having a risk of developing cancer. In a particular embodiment, the subject is suffering from Ewing sarcoma or has a risk of developing Ewing sarcoma.

Treatment or treating as used herein means an approach for obtaining beneficial or desired results, including clinical results. Beneficial or desired clinical results could include, for instance, alleviation or amelioration of one or more symptoms or conditions, diminishment of extent of cancer, stabilized state of cancer, i.e., prevent worsening, preventing spread of the cancer, delay or slowing of disease progression, amelioration or palliation of the disease state, diminishment of the reoccurrence of cancer, and remission. Treatment or treating may also prolong survival as compared to expected survival if not receiving any treatment.

Preventing or prophylaxis as used herein means an approach in which a risk of developing cancer, such as Ewing sarcoma, is reduced or prevented, including prolonging or delaying cancer development. For instance, a patient predisposed to develop a disease, such as due to genetic or hereditary predisposition, could benefit for administration of the pharmaceutical composition of the invention to prevent, reduce the risk of, delaying and/or slowing development of cancer.

In a yet further aspect of the invention there is a method for producing a pharmaceutical composition for targeted or controlled drug release. The invention relates to a diffusion method for enclosing an APIs, such as urea, or a pharmaceutically acceptable derivate of urea, such as hydroxyurea, or a pharmaceutically acceptable salt of urea, into single crystals of inorganic salts. In other words, the inorganic crystals are doped with API.

In yet further aspects of the invention there is a method 10 of manufacturing a pharmaceutical composition comprising urea doped inorganic crystals, i.e., inorganic crystals doped with urea, or a pharmaceutically acceptable derivative or salt thereof. The method comprises the steps of:
dissolving urea, or a pharmaceutically acceptable derivate or salt of thereof, in a solvent and adding an inorganic salt forming a solution, wherein the inorganic salt is selected from the group consisting of calcium carbonate, magnesium carbonate, calcium phosphate, and any combination thereof;
carbonizing the formed solution forming a carbonized solution; and
precipitating doped crystals from the carbonized solution from the carbonized solution, wherein the urea, or the pharmaceutically acceptable derivate or salt thereof, is included in the crystal lattice of the inorganic salt.

In one embodiment the method 10 comprises the steps of:
Dissolving step 11: dissolving urea, or a pharmaceutically acceptable derivate or salt thereof, in a solvent comprising Ca and/or Mg ions forming a solution;
Carbonization step 12: carbonize the formed solution forming a carbonized solution;
Precipitation step 13: allow precipitation of urea, or the pharmaceutically acceptable derivate or salt thereof doped calcium and/or magnesium crystals

In one embodiment the solvent is selected from the group consisting of calcium chloride and magnesium chloride.

In one embodiment the carbonization during the carbonization step 12 is achieved by adding carbon dioxide to the solution.

In another embodiment the method 10 comprises the steps of:
Dissolving step 11: dissolving urea, or a pharmaceutically acceptable derivate or salt thereof in a diammonium phosphate solvent and adding calcium citrate forming a solution;
Carbonization step 12: carbonize the formed solution forming a carbonized solution;
Precipitation step 13: allow precipitation of urea, or the pharmaceutically acceptable derivate or salt thereof doped calcium and/or magnesium crystals;
Washing step 14: Optionally washing the doped crystal with a washing solvent. In one embodiment the washing solvent is water and/or ethanol.

In one embodiment the pH value of the solution is maintained at 8.5 and 12 by the addition of sodium hydroxide.

The method according to the second aspect allows urea, or hydroxyurea, or a pharmaceutically acceptable derivate or salt thereof to be enclosed, in the form of a dopant, into a single crystal during the diffusion without alternating the crystallinity, crystal size, or morphology of the precipitated crystals. Examples of this can be seen in FIGS. 1B-C and FIGS. 2C and E. From the Figures it is evident that calcite crystals doped with hydroxyurea show the same morphology and crystallinity as non-doped calcite crystals, FIG. 1A and FIG. 2A.

Increasing the urea, or a pharmaceutically acceptable derivate of urea or a pharmaceutically acceptable salt of urea concentration in the synthesis results in an increased amount of drug enclosed in the crystals, see FIG. 6.

### EXAMPLES

Compositions with Hydroxyurea as Dopant

### Materials

Ammonium carbonate, ammonium acetate (HPLC degree), hydroxyurea, aspartic acid, and xanthyrol were purchased from Sigma Aldrich. Calcium chloride was purchased from Fisher scientific and hydrochloric acid (37% fuming) was purchased from Merck. Magnesium chloride, calcium citrate, diammonium phosphate, sodium hydroxide, glycine, sodium hypochlorite, hydrochloric acid (37% fuming), boric acid, o-phthalaldehyde (OPA), and sodium phosphate monobasic were purchased from Sigma Aldrich.

### Crystal Characterization

The morphology and size of the obtained crystals were studied with Scanning Electron microscopy (SEM; Zeiss Leo 1550 operated at 3 kV). The samples were prepared by dispersing the powder in ethanol and evaporating them on carbon tape. Coating with Pt/Au was done to avoid charging.

The phase composition was determined by powder X-ray Diffraction (XRD; Bruker, D8 Advanced) by using CuKα (λ=1.5418 Å) radiation. The diffractograms were recorded with a step-size of 0.05, from 20-60° (2Θ) and a step-time of 2 seconds.

### HPLC Analysis of Hydroxyurea

The quantification of hydroxyurea was done using reversed-phase high-performance liquid chromatography (RP-HPLC) with ultraviolet (UV) detection (λ=213 nm). Prior to injection on a column, the sample was mixed with 700 μl ethanol, 300 μl 1 M hydrochloric acid, and 50 μl 0.02 M Xanthyrol in 1-propanol. Hydroxyurea, with pKa 10.14, is a small molecule that does not possess UV-absorption and therefore Xanthyrol was added to act as a derivatization agent. An isocratic method was used with a mobile phase consisting of 50%20 mM ammonium acetate (pH=6.9) and 50% acetonitrile. The HPLC system was equipped with a PurospherR STAR RP-C18 column (150 mm×4.6 mm, 5 μm).

Prior to tests any excess drug non-specifically adsorbed on the particle surface was thoroughly removed using water and ethanol washing. Since hydroxyurea is highly soluble in water all excess of drug can be removed in this way.

Synthesis of Calcium Carbonate Crystals with Hydroxyurea as Dopant

The synthesis was performed with similar conditions as previously reported (Kim, Y.-Y. et al Tuning Hardness in Calcite by Incorporation of Amino acids' Nat. Mater. 2016, 15(8), 903-910). The synthesis was carried out in a closed box containing two litres of free volume. Hydroxyurea was dissolved in 20 mM CaCl₂) in order to obtain the concentrations of 400 mM and 800 mM. 40 ml of the solution was poured into a Petri dish with a surface area of 56 cm², a small magnetic stirrer was added and the Petri dish was sealed with parafilm, which were punctured with holes, allowing the precipitation to occur. Five grams of freshly crushed ammonium carbonate was added into another petri dish. Both Petri dishes were placed into the box which was placed onto a magnetic stirring plate. The synthesis was terminated after 48 hours by filtration and washing with water and ethanol. Any excess drug non-specifically adsorbed to the particle surface was thoroughly removed using water and ethanol washes prior to all tests. The formed powder was then set to dry until further analysis.

Synthesis of Magnesium Carbonate Crystals with Hydroxyurea as Dopant

The synthesis was performed with similar conditions as the previously reported (Kim, Y.-Y. et al Tuning Hardness in Calcite by Incorporation of Amino acids' Nat. Mater. 2016, 15(8), 903-910). The synthesis was carried out in a closed box containing two litres of free volume. Hydroxyurea was dissolved in 20 mM MgCl₂ in order to obtain the concentrations of 400 mM. 40 ml of the solution was poured into a Petri dish with a surface area of 56 cm², a small magnetic stirrer was added and the Petri dish was sealed with parafilm, which were punctured with holes, allowing the precipitation to occur. Five grams of freshly crushed ammonium carbonate was added into another Petri dish. Both Petri dishes were placed into the box which was placed onto a magnetic stirring plate. The synthesis was terminated after 48 hours by filtration and washing with water and ethanol. The powder was then set to dry until further analysis.

Synthesis of Hydroxyapatite Crystals with Hydroxyurea as Dopant

Hydroxyapatite was synthesized with a modified precipitation method as previously described (Wang, Z. et al ‘A facile method to in situ formation of hydroxyapatite single crystal architecture for enhanced osteoblast adhesion). Calcium citrate was added slowly (0.1 g/mL) to diammonium phosphate solution (0.0335 g/mL) containing hydroxyurea (0.02-0.07 g/mL). The pH was maintained at 8.5 and 12 by the addition of sodium hydroxide solution (400 g/L). The solution was allowed to precipitate into hydroxyapatite crystals (nanocrystals). The obtained powder was washed and bleached with sodium hypochlorite in order to remove all surface-bound hydroxyurea. After bleaching the powder was washed once again, using both water and ethanol.

### Drug Release Studies

An aliquot was taken out from the HPLC samples and mixed with 700 μl ethanol, 300 μl 1 M hydrochloric acid, and 50 μl 0.02 M xanthyrol prior to the quantification with HPLC.

Particle stability was determined by measuring hydroxyurea release after incubating in phosphate buffer (PBS solution) at pH 7.4. 50 mg of the crystals were immersed into 10 ml of phosphate buffer and placed onto a shaker, at a speed of 50 rpm. An aliquot (300 μl) was taken out after 1 hour, 1 day, 1 week, and 2 weeks. The aliquots were mixed with 700 μl ethanol, 300 μl 1 M hydrochloric acid, and 50 μl 0.02 M Xanthyrol prior to analysis with HPLC.

### Cell Culture

MCF-7 human breast cancer cells were purchased from ATCC. MCF-7 were sub-cultured in DMEM/F12 (Gibco) with 10% fetal bovine serum (FBS) and 1% penicillin/streptomycin at 37° C. in a humidified atmosphere of 5% CO₂, with complete media replacement every 48 hours. The cells were sub-cultured at 80% confluence and used within 6 passages from the thaw.

### Cell Treatment

MCF-7 cells were treated with different concentrations of calcium carbonate crystals (direct contact). Pure calcium carbonate crystals without drug were selected as a control group. The crystals were sterilized by washing in 70% ethanol, followed by resuspension in fresh cell media. Cells were treated with different concentrations of crystals and the proliferation was investigated after one and three days, respectively.

The calcite crystals were dissolved with 1 M hydrochloric acid, neutralized with 1 M sodium hydroxide, and diluted with DMEM/F12 media (1:10). The obtained particle solution was used to treat the cells and the proliferation was investigated after one and three days respectively.

Cell survival/proliferation was determined with Alamar blue assay. Cells were seeded at 1, 2 or 4×10⁴ cells/well in 96-well tissue culture treated plates. After 24 hours the media was replaced with media containing calcium carbonate crystals, doped with hydroxyurea or non-doped, dispersed over a concentration range of 100 μg/mL to 10 mg/mL. A stock solution of crystals in media, dispersed at a concentration of 10 mg/mL in PBS, was used to achieve the final treatment concentration directly before treating, by a single dilution with DMEM/F12. After incubating cells in contact with crystals for 24 or 48 hours the media was replaced with 150 μl of a 10% Alamar blue solution in fresh media. The plates were incubated at 37° C. for one hour before transferring 100 μL to a black 96-well plate. The fluorescence was detected at 570 nm excitation and 590 nm emission on a microplate reader (Infinite M200, Tekan, Switzerland).

### Results

### Example 1—Characterization of Calcium Carbonate Crystals with or without Drug

The XRD pattern showed that vaterite and calcite were both formed when no hydroxyurea was present (FIG. 1A). This was confirmed by the SEM image (FIG. 2A), where the spherical vaterite and cubic calcite crystals are clearly seen in the SEM image in FIG. 2A. The SEM images in FIGS. 2C and 2D showing samples where hydroxyurea was added only show cubic calcite crystals. In FIG. 2, FIGS. 2A and 2B represents 0 mM hydroxyurea, FIGS. 2C and 2D represents 400 mM hydoxyurea, and FIGS. 2E and 2F represents 800 mM hydroxyurea. This is confirmed by the XRD analysis, FIGS. 1A and B. The addition of hydroxyurea into the reaction restricted the formation of crystalline material only to calcite crystals (FIGS. 1B-C and FIGS. 2C-D). Due to the peak broadening in the XRD pattern, the degree of crystallinity was calculated, which showed a minor difference between the crystals with and without drug; 87% for crystals without drug and 83% for the crystals with the drug.

The crystals that were used for studying prolonged release in PBS solution, where crystals which were incubated in PBS for two weeks, showed the formation of hydroxyapatite on the surface (FIGS. 2B-F). Previous studies have shown that calcium ions will slowly dissolute from calcite, making it possible for the phosphate ions to reprecipitate hydroxyapatite on the slightly basic surface of calcite.

### Example 2—pH Studies of Doped Calcium Carbonate Crystals

The pH of the synthesis solution was measured both before and at the end of the synthesis (see Table 1 below). The pH decreased with an increasing amount of hydroxyurea while the pH after the termination of the synthesis was the same for all concentrations.

Paradoxically, while vaterite forms at room temperature and preferably in lower pH, vaterite did not form in the lower pH hydroxyurea-doped calcite solutions. This provides support to the notion that hydroxyurea may stabilize calcite, in the form of calcite against dissolution.

TABLE 1
pH measurements of starting concentration for the synthesis.

| | | |
|---|---|---|
| Concentration of Hydroxyurea | Initial pH | Final pH |

| | | | |
|---|---|---|---|
| 0 | mM | 6.99 | 9.20 |
| 400 | mM | 6.58 | 9.03 |
| 800 | mM | 6.32 | 8.97 |

### Example 3—Dissolution Studies of Doped Calcium Carbonate Crystals

The dissolution of hydroxyurea-doped calcium carbonate was investigated, visually and quantitatively, to determine which pH (1-6.5) that was sufficient to dissolve each particle concentration (0.01-50 mg/ml), thereby achieving maximal drug release. Dissolution of crystals was conducted in well-plates in acidified DMEM/F12 cell media for up to 24 hours in 37° C. For the visual investigation, images of the wells were taken with a microscope at different time points; T=0, T=1, and T=24 h. To quantify the amount of crystals dissolved, light absorbance was measured (λ=560 nm) at the same dissolution time points; T=0, T=1, and T=24 h.

The optimal pH for dissolving the calcite particle was found to be below pH 4, which resulted in almost instantaneous dissolution, FIGS. 4A-F. As can be seen in FIGS. 4A-F essentially all particles are dissolved at pH=3 and lower, at pH=4 the particles are still present but have decreased in size. Since the pH must remain close to physiological pH (7.4) for in vitro studies the rate of dissolution was determined for pH close to 7.4, FIG. 5. The amount of dissolved crystals could be quantified by measuring the absorbance of crystals at different time points and different pH. Crystals were fully dissolved after 1 hour at the concentration of 0.1 mg/mL at pH 5 and 6 as can be seen in the FIG. 4.

### Example 4—Drug Loading Efficiency of Hydroxyurea in Calcium Carbonate, Magnesium Carbonate and Hydroxyapatite Crystals

The amount of drug inside the crystals was analyzed by dissolving the calcium carbonate crystals in hydrochloric acid, which showed that increasing the drug concentration in the synthesis solutions leads to a higher amount of enclosed drug as can be seen in FIG. 6. FIG. 6 shows drug release from crystals prepared with 400 mM respectively 800 mM hydroxyurea. The lower concentration of drug during precipitation (400 mM), however, results in a higher mol % enclosed in the crystal, compared to the higher concentration (800 mM). This can be explained by the saturation of the drug in the solution, i.e., the higher starting concentration is not as efficient as the lower concentration. The amount of drug detected is presented in Table 2, together with calculations of the loading efficiency (mol %) that was calculated from the release concentration versus the starting concentration.

Drug release at pH 7.4 was conducted for two weeks for all formulations. As expected, no drug was released at pH 7.4, i.e., at physiological pH.

TABLE 2
Summary of collected data and calculations of loading
properties for the two synthesis conditions (400
mM hydroxyurea and 800 mM hydroxyurea).

| Concentration | Amount of | Amount | Loading |
|---|---|---|---|
| of hydroxyurea | drug detected | drug/calcite | efficiency |
| during synthesis | (μg) | (μg/mg) | (mol %) |
| 400 mM | 236.1 ± 25.5 | 3.6 ± 0.3 | 0.019 ± 0.002 |
| hydroxyurea | | | |
| 800 mM | 408.0 ± 46.5 | 6.7 ± 0.7 | 0.016 ± 0.002 |
| hydroxyurea | | | |

### Example 5—Cell Viability after Direct Contact During Drug Release

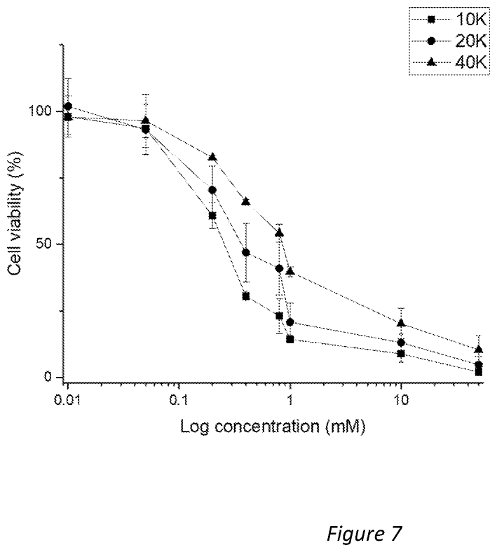

Cell viability studies are typically conducted on cells that are actively dividing, in the linear range of logarithmic division. However, tumors are typically dense, highly populated 3-dimensional cultures. Often the EC₅₀ value for a given drug differs between these two culture conditions, as lower density cultures are more susceptible to drug-induced toxicity. Therefore, hydroxyurea toxicity was validated using three different cell concentrations, 1×10⁴, 2×10⁴ and 4×10⁴ cells/well respectively of human breast cancer cells (MCF-7). FIG. 7 show the results from the EC₅₀ evaluations. The EC₅₀ value for hydroxyurea has previously been reported to be approximately 0.2 mM for breast cancer cell lines

The observed EC₅₀ values of hydroxyurea in MCF-7 cells was 0.2 mM for cells cultured at 1×10⁴/well, 0.4 mM for cells cultured at 2×10⁴/well, 0.8 mM cells cultured at 4×10⁴/well, as can be seen in FIG. 7.

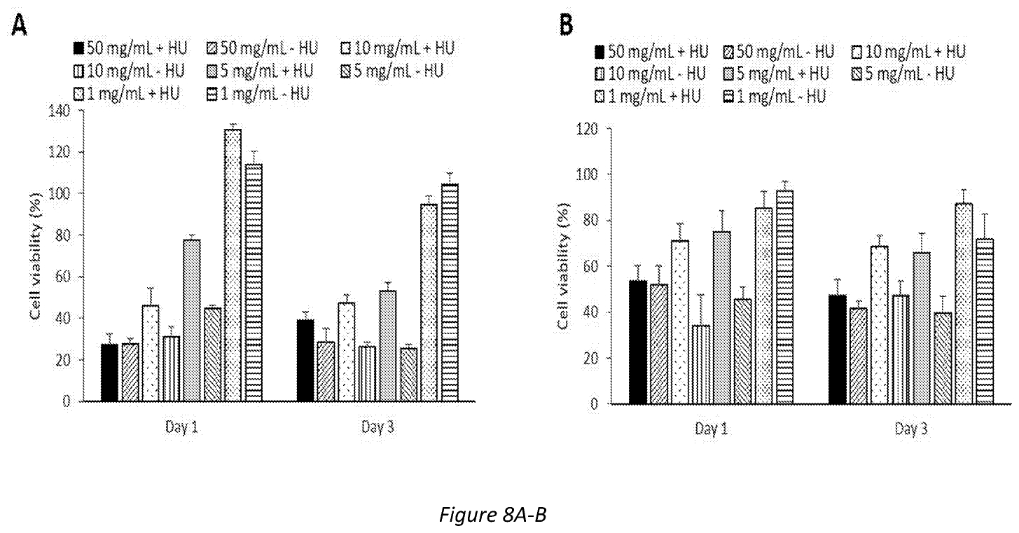

Theoretically, approximately 10 mg of crystals should be dissolved (essentially 100% release) per mL of fluid to reach the EC₅₀ for a cell concentration of 40K. The solubility of the crystals investigated in different pH media close to physiological pH, suggested that 0.1 mg/mL of crystals could be dissolved within 1 hour, FIGS. 8A-8B. Drug-loaded crystals were less toxic than non-loaded calcite. One explanation could be that drug-loaded hydroxyurea crystals are not dissolving quickly enough to release sufficient drugs at higher pH as pH=7. An additional in-vitro test was done where the synthesized crystals, with and without drug, were dissolved, neutralized, and diluted with cell media. The obtained particle solutions, 2.5 mg/mL and 5 mg/mL were tested on the cells, FIGS. 9A-9C. The result indicated that hydroxyurea affected cell proliferation, meaning that release of loaded hydroxyurea from calcium carbonate crystals, in vitro, is mostly limited by the dissolution rate of the crystal in neutral pH cell media. The lowest cell concentration (1×10⁴ cells/well) has an EC₅₀ of 200 μM hydroxyurea which translates into 2.5 mg/mL particle solution. The effect of hydroxyurea could not be observed at this concentration, i.e., same proliferation could be observed both with and without drug. A probable reason might be that the presence of high ion concentration which causes the cells to die. The highest effect of hydroxyurea can be observed in the concentration of 20.000 cells/well where a cell death of 70% was achieved after three days of treatment with a particle solution of 5 mg/mL.

Cell viability was determined with Alamar blue assay. Calcite drug-loaded crystals reduced viability by up to 50-70% as can be seen in FIG. 8A-B. However, this appears to be due to direct toxicity caused by the crystals, as non-drug loaded crystals had comparable rates of survival. Interestingly, empty calcite crystals were more toxic than drug-loaded calcite at sub-confluent cell densities (2-4×10⁴ cells/well, 1-10 mg/mL, FIGS. 9A-C). Without being bound by any theory it is likely that the calcite crystals are toxic due to their physical shape.

### Example 6—Characterization of Magnesium Carbonate Crystals with or without Drug

The amount of hydroxyurea loaded into magnesium carbonate was similar to that for calcium carbonate. Both were produced using the same manufacturing settings.

### Example 7—Characterization of Hydroxyapatite Crystals with or without Drug

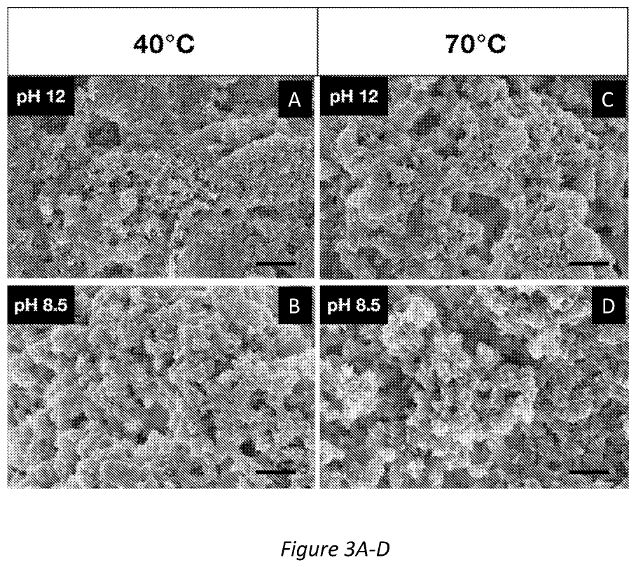

The amount of hydroxyurea loaded into hydroxyapatite was in the same range as for calcium carbonate. But higher loading pH resulted in more drug loading, about twice the amount loaded for pH 12 compared to pH 8.5. FIG. 3A-D show SEM images of the formed hydroxyurea doped hydroxyapatite crystals.

1.-9. (canceled)

10. A pharmaceutical composition comprising:
an inorganic salt; and
hydroxyurea, or a pharmaceutically acceptable salt thereof, wherein
the inorganic salt is selected from the group consisting of calcium carbonate, magnesium carbonate, calcium phosphate, and any combination thereof, and
the pharmaceutical composition is in the form of doped crystals wherein the hydroxyurea, or the pharmaceutically acceptable salt thereof, is included in a crystal lattice of the inorganic salt.

11. The pharmaceutical composition according to claim 10, wherein the pharmaceutical composition is characterized by an X-ray powder diffractogram recorded using Cu-Kα radiation, which is devoid of peaks at positions corresponding to hydroxyurea, or the pharmaceutically acceptable salt thereof.

12. The pharmaceutical composition according to claim 10, wherein the inorganic salt is selected form the group consisting of calcite, aragonite, vaterite, calcium carbonate monohydrate, magnesium calcite, magnesite, magnesium carbonate monohydrate, nesquehonite, dolomite, hydroxyapatite, β-tricalcium phosphate, α-tricalcium phosphate, calcium pyrophosphate, tetra-calcium phosphate, monetite, octo-calcium phosphate, and brushite.

13. The pharmaceutical composition according to claim 10, wherein the pharmaceutical composition comprises hydroxyurea and calcite.

14. The pharmaceutical composition according to claim 13, wherein the concentration of hydroxyurea is 2-20 μg hydroxyurea per mg calcite.

15. A method for treatment of cancer, wherein the method comprises administering a pharmaceutical composition according claim 10 to a subject suffering from cancer or having a risk of developing cancer.

16. The method according to claim 15, wherein administering the pharmaceutical composition comprises administering the pharmaceutical composition according claim 10 to a subject suffering from Ewing sarcoma or having a risk of developing Ewing sarcoma.

17. A method for producing a pharmaceutical composition, wherein the method comprises the steps of:
dissolving hydroxyurea, or a pharmaceutically acceptable salt thereof, in a solvent and adding an inorganic salt forming a solution, wherein the inorganic salt is selected from the group consisting of calcium carbonate, magnesium carbonate, calcium phosphate, and any combination thereof;
carbonizing the solution to form a carbonized solution; and
precipitating doped crystals from the carbonized solution, wherein the hydroxyurea, or the pharmaceutically acceptable salt thereof, is included in the crystal lattice of the inorganic salt.
